SEQUENCE LISTING (3 sequences)

SEQ ID NO: 1 - protein, 76 aa, Hepatitis B Virus
  variant (2)..(2): Xaa = L or R or I
LXXDWGPCXXHGXHXIRXPRTPXRVXGGVFLVDKNPHNTXESXLXVDFSQFSRGXXXVSWPKFAVPNLXSLTNLLS

SEQ ID NO: 2 - protein, 181 aa, Hepatitis B Virus
  variant (2)..(2): Xaa = N or D
SXLSWLSLDVSAAFYHXPLHPAAMPHLLXGSSGLXRYVARLSSXSXXXNXQXXXXXXLHXXCSRXLYVSLXLLYXTXGXKLHLXXHPIXLGFRKXPMGXGLSPFLLAQFTSAIXXXXXRAFXHCXXFXYMDDXVLGAXXXXHXEXLXXXXXXXLLXXGIHLNPXKTKRWGYSLNFMGYXIG

SEQ ID NO: 3 - protein, 4 aa, Hepatitis B Virus
YMDD

(19) United States
(12) Patent Application Publication
(10) Pub. No.: US 2005/0272031 A1
(43) Pub. Date: Dec. 8, 2005

(54) Viral variants and uses therefor

(75) Inventors: Thomas Bock, Tuebingen (DE); Hans Tillmann, Hannover (DE); Michael Manns, Isernhagen (DE); Christian Trautwein, Hannover (DE); Stephen Alister Locarnini, Victoria (AU); Joseph Torresi, Victoria (AU)

Correspondence Address: NIXON & VANDERHYE, PC, 901 NORTH GLEBE ROAD, 11TH FLOOR, ARLINGTON, VA, 22203, US

(73) Assignee: MELBOURNE HEALTH, Victoria (AU)

(21) Appl. No.: 11/002,999

(22) Filed: Dec. 3, 2004

Related U.S. Application Data
(62) Division of application No. 09/877,340, filed on Jun. 8, 2001, now abandoned.
(60) Provisional application No. 60/210,395, filed on Jun. 9, 2000.

(51) Int. Cl. C12Q 1/70; C12Q 1/68

(52) U.S. Cl. 435/5; 435/6

(57) ABSTRACT

Disclosed are viral variants exhibiting reduced sensitivity to particular agents including nucleoside analogues and immunological mediators such as immunoglobulins and immune cells. Also provided are hepatitis B virus (HBV) variants which exhibit a level of replication fitness in the presence of a nucleoside analogue similar to or greater than in the absence of the nucleoside analogue. The present invention also provides methods of treating HBV infection, including a method for identifying a need to change or otherwise alter an existing therapeutic regimen. Also disclosed are methods for monitoring the development in a subject of an increased HBV load in the presence of a nucleoside analogue. The present invention further provides the use of nucleoside analogue-resistant HBV variants which exhibit a similar or increased replication fitness in the presence of the nucleoside analogue compared to in the absence of the nucleoside analogue to screen for medicaments to treat HBV infection.

### BACKGROUND OF THE INVENTION

The present application claims priority from U.S. Provisional Application Ser. No. 60/210,395, filed Jun. 9, 2000, the entire contents of which is specifically incorporated herein by reference in its entirety.

### FIELD OF THE INVENTION

The present invention relates generally to viral variants exhibiting reduced sensitivity to particular agents including nucleoside analogues and immunological mediators such as immunoglobulins and immune cells. More particularly, the present invention provides hepatitis B virus (HBV) variants which exhibit a level of replication fitness in the presence of a nucleoside analogue similar to or greater than in the absence of the nucleoside analogue. The detection of such HBV variants is important in the management of therapeutic protocols including the selection of appropriate agents for treating HBV infection. In particular, the present invention contemplates a method of treating HBV infection including a method for identifying a need to change or otherwise alter an existing therapeutic regimen. Furthermore, the method of treatment further encompasses selecting an anti-viral agent or combination of anti-viral agents which would be less likely to result in development of resistance to anti-viral therapy. The method of this aspect of the present invention is predicated in part on monitoring the development in a subject of an increased HBV load in the presence of a nucleoside analogue. This may be manifested by an increase in HBV DNA levels compared to levels in patients prior to treatment. The recognition of such increased viral load and/or DNA levels is indicative of the development of a variant HBV resistant to said nucleoside analogue and an immune mediated response. The clinician is then able to modify an existing treatment protocol or select an appropriate treatment protocol accordingly. The present invention further provides the use of nucleoside analogue-resistant HBV variants which exhibit a similar or increased replication fitness in the presence of the nucleoside analogue compared to in the absence of the nucleoside analogue to screen for medicaments to treat HBV infection.

### BACKGROUND OF THE INVENTION

Bibliographic details of the publications numerically referred to in this specification are collected at the end of the description.

Specific mutations in an amino acid sequence are represented herein as “Xaa₁nXaa₂” where Xaa₁ is the original amino acid residue before mutation, n is the residue number and Xaa₂ is the mutant amino acid. The abbreviation “Xaa” may be the three letter or single letter (i.e., “X”) code. The amino acid residues for Hepatitis B virus DNA polymerase are numbered with the residue methionine in the motif Tyr Met Asp Asp (YMDD) being residue number 550. The amino acid residues for hepatitis B virus surface antigen are number according to Norder et al. (15).

Hepatitis B virus (HBV) can cause debilitating disease conditions and can lead to acute liver failure. HBV is a DNA virus which replicates via an RNA intermediate and utilizes reverse transcription in its replication strategy (1). The HBV genome is of a complex nature having a partially double stranded DNA structure with overlapping open reading frames encoding surface, core, polymerase and X genes. The complex nature of the HBV genome is represented in FIG. 1.

The presence of an HBV DNA polymerase has led to the proposition that nucleoside analogues could act as effective anti-viral agents. Examples of nucleoside analogues currently being tested are penciclovir and its oral form famciclovir (2,3,4,5), lamivudine[(−)-β-2′-deoxy-3′-thiacytidine; “3TC” or “LAM”] (6,7). Adefovir has been shown to have effective anti-HBV activity in vitro. Generally, the nucleotide analogues are used in conjunction with hepatitis B immunoglobulin (HBIG) therapy in the transplant setting. Interferon is currently used in the treatment of chronic HBV infection.

Lamivudine is a particularly potent inhibitor of HBV replication and reduces HBV DNA titres in the sera of chronically infected patients after orthotopic liver transplantation (OLT) by inhibiting viral DNA synthesis. OLT is a therapeutic option for end-stage liver disease. Because of HBV re-infection, results of liver transplantation for HBV-related end-stage are frequently poor. In work leading up to the present invention, the inventors observed the emergence of nucleoside analogue resistant HBV variants. The detection of such variants provides an important aspect of developing and monitoring therapeutic protocols against HBV infection. Aspects of the present invention have been disclosed in Tillmann et al. (12) which is incorporated herein by reference.

### SUMMARY OF THE INVENTION

Throughout this specification, unless the context requires otherwise, the word “comprise,” or variations such as “comprises” or “comprising,” will be understood to imply the inclusion of a stated element or integer or group of elements or integers but not the exclusion of any other element or integer or group of elements or integers.

One aspect of the present invention contemplates an HBV variant exhibiting a replication fitness, in the presence of a nucleoside analogue, similar to or greater than in the absence of said nucleoside analogue.

Another aspect of the present invention provides an HBV variant carrying a mutation in the nucleoside sequence encoding a DNA polymerase resulting in an amino acid addition, substitution and/or deletion in said DNA polymerase in one or more amino acids as set forth in Formulae I and/or II:

FORMULA I

L, B₁, B₂, D, W, G, P, C, B₃, B₄, H, G, B₅, H, B₆,
I, R, B₇, P, R, T, P, B₈, R, V, B₉, G, G, V, F, L,
D, K, N, P, H, N, T, B₁₀, E, S, B₁₁, L, B₁₂, V, D,
F, S, Q, F, S, R, G, B₁₃, B₁₄, B₁₅, V, S, W, P, K,
F, A, V, P, N, L, B₁₆, S, L, T, N, L, L, S*

wherein:
B₁ is L, or R, or I
B₂ is E, or D
B₃ is T, or D, or A, or N, or Y
B₄ is E, or D
B₅ is E, or K, or Q
B₆ is H, or R, or N,
B₇ is I, or T
B₈ is A; or S
B₉ is T or R
B₁₀ is A, or T, or S
B₁₁ is R, or T
B₁₂ is V, or G
B₁₃ is S, or I, or T, or N, or V
B₁₄ is T, or S, or H, or Y
B₁₅ is R, or H, or K, or Q
B₁₆ is Q, or P;

and

FORMULA II

S Z₁ L S W L S L D V S A A F Y H Z₂ P L H P A A M
P H L L Z₃ G S S G L Z₄ R Y V A R L S S Z₅ S Z₆ Z₇
X N Z₈ Q Z₉ Z₁₀ X X X Z₁₁ L H Z₁₂ Z₁₃ C S R Z₁₄ L
Y V S L Z₁₅ L L Y Z₁₆ T Z₁₇ G Z₁₈ K L H L Z₁₉ Z₂₀
H P I Z₂₁ L G F R K Z₂₂ P M G Z₂₃ G L S P F L L A
Q F T S A I Z₂₄ Z₂₅ Z₂₆ Z₂₇ Z₂₈ R A F Z₂₉ H C Z₃₀
Z₃₁ F Z₃₂ Y M* D D Z₃₃ V L G A Z₃₄ Z₃₅ Z₃₆ Z₃₇ H
Z₃₈ E Z₃₉ L Z₄₀ Z₄₁ Z₄₂ Z₄₃ Z₄₄ Z₄₅ Z₄₆ L L Z₄₇
Z₄₈ G I H L N P Z₄₉ K T K R W G Y S L N F M G Y
Z₅₀ I G

wherein:
X is any amino acid;
Z₁ is N or D;
Z₂ is I or P;
Z₃ is I or V;
Z₄ is S or D;
Z₅ is T or N;
Z₆ is R or N;
Z₇ is N or I;
Z₈ is N or Y or H;
Z₉ is H or Y;
Z₁₀ is G or R;
Z₁₁ is D or N;
Z₁₂ is D or N;
Z₁₃ is S or Y;
Z₁₄ is N or Q;
Z₁₅ is L or M;
Z₁₆ is K or Q;
Z₁₇ is Y or F;
Z₁₈ is R or W;
Z₁₉ is Y or L;
Z₂₀ is S or A;
Z₂₁ is I or V;
Z₂₂ is I or L;
Z₂₃ is V or G;
Z₂₄ is C or L;
Z₂₅ is A or S;
Z₂₆ is V or M;
Z₂₇ is V or T;
Z₂₈ is R or C;
Z₂₉ is For P;
Z₃₀ is L or V;
Z₃₁ is A or V;
Z₃₂ is S or A;
Z₃₃ is V or L or M;
Z₃₄ is K or R;
Z₃₅ is S or T;
Z₃₆ is V or G;
Z₃₇ is Q or E;
Z₃₈ is L or S or R;
Z₃₉ is S or F;
Z₄₀ is F or Y;
Z₄₁ is T or A;
Z₄₂ is A or S;
Z₄₃ is V or I;
Z₄₄ is T or C;
Z₄₅ is N or S;
Z₄₆ is F or V;
Z₄₇ is S or D;
Z₄₈ is L or V;
Z₄₉ is N or Q;
Z₅₀ is V or I; and
M* is amino acid 550
   and wherein S* in Formula I is designated as amino acid 420 and the first S in Formula II is designated as amino acid 421;
   and wherein said variant exhibits a replication fitness in the presence of a nucleoside analogue similar to or greater than in the absence of said nucleoside analogue.

Yet another aspect of the present invention is directed to an HBV variant comprising a mutation in the nucleotide sequence encoding the HBV surface antigen which results in an amino acid addition, substitution and/or deletion in said surface antigen in a region corresponding to the amino acid sequences set forth in Formulae I and/or II wherein said variant exhibits a replication fitness in the presence of a nucleoside analogue similar to or greater than in the absence of said nucleoside analogue.

Even yet another aspect of the present invention is directed to an HBV variant comprising a mutation in the nucleotide sequence encoding the HBV surface antigen which results in an amino acid addition, substitution and/or deletion in said surface antigen in a region corresponding to the amino acid sequences set forth in Formulae I and/or II wherein said variant results in HBV DNA levels in the presence of a nucleoside analogue similar to or greater than the levels detected in pretreated patients.

Still yet another aspect of the present invention provides an HBV comprising a mutation in the nucleotide sequences encoding a DNA polymerase and a mutation in the nucleotide sequences encoding the surface antigen wherein each mutation results in an amino acid addition, substitution and/or deletion to each of the DNA polymerase and surface antigen and wherein said variants exhibits a replication fitness in the presence of a nucleoside analogue similar to or greater than in the absence of said nucleoside analogue.

Another aspect of the present invention contemplates a method for determining whether an HBV strain exhibits reduced sensitivity to a nucleoside analogue, said method comprising isolating DNA or corresponding mRNA from said HBV and screening for a mutation in the nucleotide sequence encoding the DNA polymerase and optionally the surface antigen (listed below in parenthesis) wherein the presence of a T474N(P120T), M550V (1195M), M550I (W196S), L526M, W499S/W499Q (G145R) mutation, or combinations thereof or an equivalent one or more other mutation is indicative of a variant wherein said variant exhibits a replication fitness in the presence of a nucleoside analogue similar to or greater than in the absence of said nucleoside analogue.

Another aspect of the present invention contemplates a method for detecting an HBV agent which exhibits inhibitory activity to an HBV, said method comprising:
generating a genetic construct comprising a replication competent-effective amount of the genome from said HBV contained in a plasmid vector and then transfecting said cells with said construct;
contacting said cells, before, during and/or after transfection, with the agent to be tested;
culturing said cells for a time and under conditions sufficient for the HBV to replicate, express genetic sequences and/or assemble and/or release virus or virus-like particles if resistant to said agent; and
subjecting the cells, cell lysates or culture supernatant fluid to viral- or viral-component-detection means to determine whether or not the virus has replicated, expressed genetic material and/or assembled and/or been released in the presence of said agent.

Still another aspect of the present invention provides a method for detecting an HBV agent which exhibits inhibitory activity to an HBV, said method comprising:
generating a genetic construct comprising a replication competent-effective amount of the genome from said HBV contained in or fused to an amount of a baculovirus genome effective to infect cells and then infecting said cells with said construct;
contacting said cells, before, during and/or after infection, with the agent to be tested;
culturing said cells for a time and under conditions sufficient for the HBV to replicate, express genetic sequences and/or assemble and/or release virus or virus-like particles if resistant to said agent; and
subjecting the cells, cell lysates or culture supernatant fluid to viral- or viral-component-detection means to determine whether or not the virus has' replicated, expressed genetic material and/or assembled and/or been released in the presence of said agent.

Still another aspect of the present invention provides a method for detecting an HBV agent which exhibits inhibitory activity to an HBV, said method comprising:
generating a continuous cell line comprising an infectious copy of the genome of said HBV in a replication competent effective amount such that said infectious HBV genome is stably integrated into said continuous cell line such as but not limited to 2.2.15 or AD;
contacting said cells with the agent to be tested;
culturing said cells for a time and under conditions sufficient for the HBV to replicate, express genetic sequences and/or assemble and/or release virus or virus-like particles if resistant to said agent; and
subjecting the cells, cell lysates or culture supernatant fluid to viral- or viral-component-detection means to determine whether or not the virus has replicated, expressed genetic material and/or assembled and/or been released in the presence of said agent.

Yet another aspect of the present invention contemplates a method of treating a patient infected with HBV, said method comprising administering to said patient an effective amount of a nucleoside analogue sufficient initially to inhibit HBV replication, monitoring HBV levels to ascertain the presence of an increased viral load in the presence of said nucleoside analogue and then changing the therapeutic protocol to permit inhibition of HBV levels.

Still yet another aspect of the present invention provides a method of treating a subject infected with HBV, said method comprising administering to said subject an effective amount of LAM or its chemical derivatives or homologues or a functionally related nucleoside analogue for a time and under conditions sufficient for the development of HBV variants which exhibit a level of fitness to said nucleoside analogue similar to or greater than in the absence of said nucleoside analogue which variant is resistant to HBIG or its equivalent and then altering the therapeutic protocol to enable the inhibition of replication of the HBV variants.

Another aspect of the present invention provides a method of treating a subject infected with HBV, said method comprising administering to said subject an anti-HBV agent or combination of agents which after prolonged exposure to said HBV does not select for HBV variants which exhibit a level of replication fitness similar to or greater than in the absence of said nucleoside analogue.

Yet another aspect of the present invention extends to the use of an HBV variant which has a level of replication fitness in the presence of a nucleoside analogue similar to or greater than in the absence of said nucleoside analogue in the detection of an anti-viral agent capable of inhibiting the replication of said HBV variant.

Still yet another aspect, the invention contemplates a computer program product for assessing the likely usefulness of a viral variant or biological sample comprising same for determining an appropriate therapeutic protocol in a subject, said product comprising:
(1) code that receives as input Ivs for at least two features associated with said viral agents or biological sample comprising same, wherein said features are selected from:
   (a) the ability to exhibit resistance for reduced sensitivity to a particular compound or immunological agent;
   (b) an altered DNA polymerase from wild-type HBV;
   (c) an altered surface antigen from wild-type HBV; or
   (d) morbidity or recovery potential of a patient;
(2) code that adds said Iᵥs to provide a sum corresponding to a Pᵥ for said viral variants or biological samples; and
(3) a computer readable medium that stores the codes.

In a related aspect, the invention extends to a computer for assessing the likely usefulness of a viral variant or biological sample comprising same in a subject, wherein said computer comprises:
(1) a machine-readable data storage medium comprising a data storage material encoded with machine-readable data, wherein said machine-readable data comprise Iᵥs for at least two features associated with said viral variant or biological sample; wherein said features are selected from:
   (a) the ability to exhibit resistance for reduced sensitivity to a particular compound or immunological agent;
   (b) an altered DNA polymerase from wild-type HBV;
   (c) an altered surface antigen from wild-type HBV; or
   (d) morbidity or recovery potential of a patient;
(2) a working memory for storing instructions for processing said machine-readable data;
(3) a central-processing unit coupled to said working memory and to said machine-readable data storage medium, for processing said machine readable data to provide a sum of said Iᵥs corresponding to a Pᵥ for said compound(s); and
(4) an output hardware coupled to said central processing unit, for receiving said Pᵥ.

### BRIEF DESCRIPTION OF THE DRAWINGS

The following drawings form part of the present specification and are included to further demonstrate certain aspects of the present invention. The invention may be better understood by reference to the following description taken in conjunction with the accompanying drawings, in which like reference numerals identify like elements, and in which:

FIG. 1 is a diagrammatic representation showing the partially double stranded DNA HBV genome showing the overlapping open reading frames encoding surface (S), core (C), polymerase (P) and X gene.

FIG. 2 is a schematic diagram of HBV replication competent vectors containing HBV polymerase mutants G145R and P120T (serotype HBV adw2; Acc. No. X02763) are escape mutants in the “a”-determinant of the S-gene. Corresponding polymerase gene mutations comprise G145R=W499S or W499Q and P120T=T474N. The lamivudine associated mutations found after liver transplantation were Type I: L526M/M550V and Type II: M550I. The corresponding substituted S-gene mutations were M550V=1195M, M5501=W196S. Double and triple mutations were found in liver transplanted patients after HBIG and nucleoside analogues treatment.

FIG. 3 is a photographic representation showing the levels of HBV progeny DNA. Replicative HBV intermediates were isolated from HuH-7/HepG2 cells 5 to 6 days after infection. Southern Blot analysis revealed decreased progeny DNA levels of LAM/FCV associated mutants in comparison to wild-type HBV constructs.

FIG. 4 is a photographic representation showing replication levels of polymerase mutants under LAM treatment in cell culture experiments. The addition of 0.5 up to 5 μM LAM to the cell culture medium demonstrated sensitivity to LAM for the HBIG-mutants while an enhanced increased in replication fitness was observed for the HBIG/LAM-mutants.

FIG. 5 is a photographic representation of total HBV and RNA levels of the polymerase mutants compared to wild-type HBV constructs. Total RNA was isolated from transfected cells. Northern Blot analysis revealed comparable amounts of mutant RNA to wild-type HBV RNA.

FIG. 6 is a photographic representation of encapsidated RNA levels of polymerase mutants revealing similar amounts compared to wild-type HBV constructs. The analysis of the encapsidated pregenomic RNA levels revealed comparable amounts of mutant RNAs to wild-type HBV RNAs. These data and the results of total RNA demonstrated that the levels of viral RNAs were not responsible for the progeny DNA levels of the polymerase mutants.

FIG. 7 is a photographic representation showing polymerase efficacy of polymerase mutants determined by endogenous polymerase assay. The results confirm the progeny DNA findings and showed that the HBIG/LAM associated combination mutations were no longer sensitive to LAM but revealed an enhanced polymerase activity when harvested from cells following treatment with LAM.

FIG. 8 is a photographic representation showing regulation of HBV DNA (+)-strand synthesis isolated from encapsidated HBV DNA. The last step in the viral life cycle before budding to the ER and secretion is the generation of the viral (+)-stand. A complete (+)-strain is a benefit for secretion of the virus and may be due to a higher viral load in the patients' serum. Isolated progeny DNA following Southern Blot using a strand specific HBV probe demonstrated an increased (+) strand level for the LAM and HBIG/LAM combination mutants in comparison to wild-type HBV.

FIG. 9 is a diagrammatic representation of a system used to carry out the instructions encoded by the storage medium.

FIG. 10 is a diagrammatic representation of a cross-section of a magnetic storage medium.

FIG. 11 is a diagrammatic representation of a cross-section of an optically readable data storage system.

ABBREVIATIONS

| ABBREVIATION | DESCRIPTION |
|---|---|
| LAM | lamivudine |
| 3TC | (LAM); (−)-β-2′-deoxy-3′-thiacytidine |
| HBIG | Hepatitis B immunoglobulin |
| HBV | Hepatitis B virus |
| ER | Endoplasmic reticulum |

### DESCRIPTION OF ILLUSTRATIVE EMBODIMENTS

Lamivudine (LAM or 3TC) is a potent inhibitor of HBV replication. It is observed that HBV DNA titres are reduced in the sera of chronically infected patients after OLT and treatment with LAM and HBIG. LAM inhibits viral DNA synthesis. However, after a few months, there is an increase in HBV titres. Levels rose to pre-treatment levels. In accordance with the present invention, the inventors sequenced the genomes of the HBV resistant variants and revealed a number of mutations in the HBV polymerase gene which resulted in a level of replication fitness in the presence of a nucleoside analogue relative to its absence. Such a phenomenon is demonstratable by detecting viral load or burden in patients exposed to the nucleoside analogue. Viral load or burden is conveniently determined by detecting viral nucleic acid molecules (e.g., DNA), replicative intermediates, polymerase activity, levels of surface antigen and/or titre of viral particles. The detection of such replication fit HBV variants in the presence of a nucleoside analogue is an important step in determining an appropriate therapeutic protocol for patients.

Accordingly, one aspect of the present invention contemplates an HBV variant exhibiting a replication fitness in the presence of a nucleoside analogue similar to or greater than in the absence of said nucleoside analogue.

Preferably, the HBV variant carries a mutation in the nucleotide sequence encoding the HBV DNA polymerase. Such mutation results in an addition, substitution and/or deletion of an amino acid sequence of the DNA polymerase. Reference to the HBV DNA polymerase includes domains F and A through E set forth in Formula I below:

FORMULA I

L, B₁, B₂, D, W, G, P, C, B₃, B₄, H, G, B₅, H, B₆,
I, R, B₇, P, R, T, P, B₈, R, V, B₉, G, G, V, F, L,
V, D, K, N, P, H, N, T, B₁₀, E, S, B₁₁, L, B₁₂, V,
D, F, S, Q, F, S, R, G, B₁₃, B₁₄, B₁₅, V, S, W, P,
K, F, A, V, P, N, L, B₁₆, S, L, T, N, L, L, S*

wherein:
B₁ is L, or R, or I
B₂ is E, or D
B₃ is T, or D, or A, or N, or Y
B₄ is E, or D
B₅ is E, or K, or Q
B₆ is H, or R, or N,
B₇ is I, or T
B₈ is A, or S
B₉ is T or R
B₁₀ is A, or T, or S
B₁₁ is R, or T
B₁₂ is V, or G
B₁₃ is S, or I, or T, or N, or V
B₁₄ is T, or S, or H, or Y
B₁₅ is R, or H, or K, or Q
B₁₆ is Q, or P;
   and wherein S* is designated as amino acid 420.

In this specification, reference is particularly made to the conserved regions as defined by Poch et al. (16) as domains A to E (see also reference 17). Regions A to E are defined by the amino acid sequence set forth in Formula II below:

FORMULA II

S Z₁ L S W L S L D V S A A F Y H Z₂ P L H P A A M
P H L L Z₃ G S S G L Z₄ R Y V A R L S S Z₅ S Z₆ Z₇
X N Z₈ Q Z₉ Z₁₀ X X X Z₁₁ L H Z₁₂ Z₁₃ C S R Z₁₄ L
Y V S L Z₁₅ L L Y Z₁₆ T Z₁₇ G Z₁₈ K L H L Z₁₉ Z₂₀
H P I Z₂₁ L G F R K Z₂₂ P M G Z₂₃ G L S P F L L A
Q F T S A I Z₂₄ Z₂₅ Z₂₆ Z₂₇ Z₂₈ R A F Z₂₉ H C Z₃₀
Z₃₁ F Z₃₂ Y M* D D Z₃₃ V L G A Z₃₄ Z₃₅ Z₃₆ Z₃₇ H
Z₃₈ E Z₃₉ L Z₄₀ Z₄₁ Z₄₂ Z₄₃ Z₄₄ Z₄₅ Z₄₆ L L Z₄₇
Z₄₈ G I H L N P Z₄₉ K T K R W G Y S L N F M G Y
Z₅₀ I G

wherein:
X is any amino acid;
Z₁ is N or D;
Z₂ is I or P;
Z₃ is I or V;
Z₄ is S or D;
Z₅ is T or N;
Z₆ is R or N;
Z₇ is N or I;
Z₈ is N or Y or H;
Z₉ is H or Y;
Z₁₀ is G or R;
Z₁₁ is D or N;
Z₁₂ is D or N;
Z₁₃ is S or Y;
Z₁₄ is N or Q;
Z₁₅ is L or M;
Z₁₆ is K or Q;
Z₁₇ is Y or F;
Z₁₈ is R or W;
Z₁₉ is Y or L;
Z₂₀ S or A;
Z₂₁ is I or V;
Z₂₂ is I or L;
Z₂₃ is V or G;
Z₂₄ is C or L;
Z₂₅ is A or S;
Z₂₆ is V or M;
Z₂₇ is V or T;
Z₂₈ is R or C;
Z₂₉ is F or P;
Z₃₀ is L or V;
Z₃₁ is A or V;
Z₃₂ is S or A;
Z₃₃ is V or L or M;
Z₃₄ is K or R;
Z₃₅ is S or T;
Z₃₆ is V or G;
Z₃₇ is Q or E;
Z₃₈ is L or S or R;
Z₃₉ is S or F;
Z₄₀ is F or Y;
Z₄₁ is T or A;
Z₄₂ is A or S;
Z₄₃ is V or I;
Z₄₄ is T or C;
Z₄₅ is N or S;
Z₄₆ is F or V;
Z₄₇ is S or D;
Z₄₈ is L or V;
Z₄₉ is N or Q;
Z₅₀ is V or I; and
M* is amino acid 550
   and wherein the first S is designated as amino acid 421.

According, another aspect of the present invention provides an HBV variant carrying a mutation in the nucleoside sequence encoding a DNA polymerase resulting in an amino acid addition, substitution and/or deletion in said DNA polymerase in one or more amino acids as set forth in Formulae I and/or II:

FORMULA I

L, B₁, B₂, D, W, G, P, C, B₃, B₄, H, G, B₅, H, B₆,
I, R, B₇, P, R, T, P, B₈, R, V, B₉, G, G, V, F, L,
V, D, K, N, P, H, N, T, B₁₀, E, S, B₁₁, L, B₁₂, V,
D, F, S, Q, F, S, R, G, B₁₃, B₁₄, B₁₅, V, S, W, P,
K, F, A, V, P, N, L, B₁₆, S, L, T, N, L, L, S*

wherein:
B₁ is L, or R, or I
B₂ is E, or D
B₃ is T, or D, or A, or N, or Y
B₄ is E, or D
B₅ is E, or K, or Q
B₆ is H, or R, or N,
B₇ is I, or T
B₈ is A, or S
B₉ is T or R
B₁₀ is A, or T, or S
B₁₁ is R, or T
B₁₂ is V, or G
B₁₃ is S, or I, or T, or N, or V
B₁₄ is T, or S, or H, or Y
B₁₅ is R, or H, or K, or Q
B₁₆ is Q, or P;

and

FORMULA II

S Z₁ L S W L S L D V S A A F Y H Z₂ P L H P A A M
P H L L Z₃ G S S G L Z₄ R Y V A R L S S Z₅ S Z₆ Z₇
X N Z₈ Q Z₉ Z₁₀ X X X Z₁₁ L H Z₁₂ Z₁₃ C S R Z₁₄ L
Y V S L Z₁₅ L L Y Z₁₆ T Z₁₇ G Z₁₈ K L H L Z₁₉ Z₂₀
H P I Z₂₁ L G F R K Z₂₂ P M G Z₂₃ G L S P F L L A
Q F T S A I Z₂₄ Z₂₅ Z₂₆ Z₂₇ Z₂₈ R A F Z₂₉ H C Z₃₀
Z₃₁ F Z₃₂ Y M* D D Z₃₃ V L G A Z₃₄ Z₃₅ Z₃₆ Z₃₇ H
Z₃₈ E Z₃₉ L Z₄₀ Z₄₁ Z₄₂ Z₄₃ Z₄₄ Z₄₅ Z₄₆ L L Z₄₇
Z₄₈ G I H L N P Z₄₉ K T K R W G Y S L N F M G Y
Z₅₀ I G

wherein:
X⁻ is any amino acid;
Z₁ is N or D;
Z₂ is I or P;
Z₃ is I or V;
Z₄ is S or D;
Z₅ is T or N;
Z₆ is R or N;
Z₇ is N or I;
Z₈ is N or Y or H;
Z₉ is H or Y;
Z₁₀ is G or R;
Z₁₁ is D or N;
Z₁₂ is D or N;
Z₁₃ is S or Y;
Z₁₄ is N or Q;
Z₁₅ is L or M;
Z₁₆ is K or Q;
Z₁₇ is Y or F;
Z₁₈ is R or W;
Z₁₉ is Y or L;
Z₂₀ is S or A;
Z₂₁ is I or V;
Z₂₂ is I or L;
Z₂₃ is V or G;
Z₂₄ is C or L;
Z₂₅ is A or S;
Z₂₆ is V or M;
Z₂₇ is V or T;
Z₂₈ is R or C;
Z₂₉ is For P;
Z₃₀ is L or V;
Z₃₁ is A or V;
Z₃₂ is S or A;
Z₃₃ is V or L or M;
Z₃₄ is K or R;
Z₃₅ is S or T;
Z₃₆ is V or G;
Z₃₇ is Q or E;
Z₃₈ is L or S or R;
Z₃₉ is S or F;
Z₄₀ is F or Y;
Z₄₁ is T or A;
Z₄₂ is A or S;
Z₄₃ is V or I;
Z₄₄ is T or C;
Z₄₅ is N or S;
Z₄₆ is F or V;
Z₄₇ is S or D;
Z₄₈ is L or V;
Z₄₉ is N or Q;
Z₅₀ is V or I; and
M* is amino acid 550
   and wherein S* in Formula I is designated as amino acid 420 and the first S in Formula II is designated as amino acid 421;
   and wherein said variant exhibits a replication fitness in the presence of a nucleoside analogue similar to or greater than in the absence of said nucleoside analogue.

Preferred nucleoside analogues, including FAM and/or LAM and their chemical derivatives and homologues, are those which select mutations in the B and/or C domains of HBV polymerase.

Furthermore, in one particular embodiment, the nucleoside analogue selects a corresponding mutation in the HBV surface antigen gene resulting in an HBIG-resistant mutant. In another particularly preferred embodiment, the replication fitness HBV variant is selected following exposure to both the nucleoside analogue and HBIG treatment.

Accordingly, another aspect of the present invention is directed to an HBV variant comprising a mutation in the nucleotide sequence encoding the HBV surface antigen which results in an amino acid addition, substitution and/or deletion in said surface antigen in a region corresponding to the amino acid sequences set forth in Formulae I and/or II wherein said variants exhibits a replication fitness in the presence of a nucleoside analogue similar to or greater than in the absence of said nucleoside analogue.

In a related embodiment of the present invention, there is provided an HBV variant comprising a mutation in the nucleotide sequence encoding the HBV surface antigen which results in an amino acid addition, substitution and/or deletion in said surface antigen in a region corresponding to the amino acid sequences set forth in Formulae I and/or II wherein said variant results in HBV DNA levels in the presence of a nucleoside analogue similar to or greater than the levels detected in pretreated patients.

More particularly, the present invention provides an HBV comprising a mutation in the nucleotide sequences encoding a DNA polymerase and a mutation in the nucleotide sequences encoding the surface antigen wherein each mutation results in an amino acid addition, substitution and/or deletion to each of the DNA polymerase and surface antigen and wherein said variants exhibits a replication fitness in the presence of a nucleoside analogue similar to or greater than in the absence of said nucleoside analogue.

Preferred mutations leading to a nucleoside analogue mediated replication fitness HBV variant include but are not limited to mutants selected for directly by the nucleoside analogue (e.g., LAM) as well as those selected by other agents such as HBIG. An example of the latter is G145R and P120T. An example of the former is M550I, M550V and L526M or various combinations thereof. Most preferably, the mutants are selected by exposure to a combination of both LAM and HBIG.

Particularly preferred mutants encompassed by the present invention include but are not limited to G145R, M550I, P120T, M550V and L526M or various combinations thereof such as M550I+P120T and L526M+M550V+P120T.

The identification of the replication fitness HBV variants of the present invention provides a means of screening for and identifying anti-viral agents for use in alternative therapeutic strategies.

Accordingly, yet another aspect of the present invention contemplates a method for determining whether an HBV strain exhibits reduced sensitivity to a nucleoside analogue, said method comprising isolating DNA or corresponding mRNA from said HBV and screening for a mutation in the nucleotide sequence encoding the DNA polymerase and optionally the surface antigen (indicated below in parenthesis) wherein the presence of a T474N(P120T), M550V (1195M), M550I (W196S), L526M, W499S/W499Q (G145R) mutation or various combinations thereof or an equivalent one or more other mutation is indicative of a variant wherein said variants exhibits a replication fitness in the presence of a nucleoside analogue similar to or greater than in the absence of said nucleoside analogue.

Furthermore, the present invention extends to the use of an HBV variant which has a level of replication fitness in the presence of a nucleoside analogue similar to or greater than in the absence of said nucleoside analogue in the detection of an anti-viral agent capable of inhibiting the replication of said HBV variant.

The term “inhibiting the replication” includes inhibiting one or more stages of infection including replication, assembly and/or release of HBV including any intermediary steps during the process of viral infection, replication assembly and/or release.

The present invention is predicated in part on the ability to screen for HBV variants having enhanced replication fitness in the presence of a nucleoside analogue. This may be accomplished in any number of ways such as determining viral load or burden, viral titre or detecting indicators such as nucleic acid levels and HBV antigenic determinants. Furthermore, the present invention permits the development of assays to screen for HBV variants exhibiting enhanced replication fitness or for agents useful in therapy against such variants.

Conveniently, this aspect of the present invention is practised using a plasmid vector system or a baculovirus vector system.

Accordingly, another aspect of the present invention is directed to a method of detecting an HBV variant exhibiting a level of replication fitness in the presence of a nucleoside analogue similar to or greater than the replication fitness in the absence of said nucleoside analogue, said method comprising:
generating a genetic construct comprising a replication competent-effective amount of the genome from an HBV contained in or fixed to an amount of a plasmid vector and then transfecting said cells with said construct;
contacting said cells before, during or after transfection with a nucleoside analogue capable of inhibiting the replication of a wild-type HBV;
culturing said cells for a time and under conditions sufficient for the HBV to replicate, express genetic sequences and/or assemble and/or release virus or virus-like particles if exhibiting replication fitness in the presence of said nucleoside analogues; and
subjecting the cells, cell lysates or culture supernatant fluid to viral- or viral-component detection means to determine levels of the replication, expression of genetic material and/or assembly and/or release.

In an alternative embodiment, the present invention is directed to a method of detecting an HBV variant exhibiting a level of replication fitness in the presence of a nucleoside analogue similar to or greater than the replication fitness in the absence of said nucleoside analogue, said method comprising:—
generating a genetic construct comprising a replication competent-effective amount of the genome from an HBV contained in or fixed to an amount of a baculovirus genome effective to infect cells and then infecting said cells with said construct;
contacting said cells before, during or after infection with a nucleoside analogue capable of inhibiting the replication of a wild-type HBV;
culturing said cells for a time and under conditions sufficient for the HBV to replicate, express genetic sequences and/or assemble and/or release virus or virus-like particles if exhibiting replication fitness in the presence of said nucleoside analogues; and
subjecting the cells, cell lysates or culture supernatant fluid to viral- or viral-component detection means to determine levels of the replication, expression of genetic material and/or assembly and/or release.

In a further alternative embodiment of the present invention, there is provided a method for detecting an HBV agent which exhibits inhibitory activity to an HBV, said method comprising:
generating a continuous cell line comprising an infectious copy of the genome of said HBV in a replication competent effective amount such that said infectious HBV genome is stably integrated into said continuous cell line such as but not limited to 2.2.15 or AD;
contacting said cells with the agent to be tested;
culturing said cells for a time and under conditions sufficient for the HBV to replicate, express genetic sequences and/or assemble and/or release virus or virus-like particles if resistant to said agent; and
subjecting the cells, cell lysates or culture supernatant fluid to viral- or viral-component-detection means to determine whether or not the virus has replicated, expressed genetic material and/or assembled and/or been released in the presence of said agent.

Conveniently, but not necessarily, the method is conducted with suitable controls such as culturing the cells in the absence of the nucleoside analogue.

The preferred nucleoside analogue is LAM and/or FAM but the present invention extends to derivatives and homologues of LAM and/or FAM as well as other functionally related nucleoside analogues as well as agents selected by the above methods.

The detection of HBV or its components in cells, cell lysates and culture supernatant fluid may be by any convenient means. For example, total HBV DNA or RNA may be determined, replicative intermediates may be detected or HBV-specific products or gene transcripts may be determined. Suitable assay means include PCR, PCR sequencing, nucleic acid hybridization protocols such as Northern Blots, Southern Blots and in situ hybridization and antibody procedures such as ELISA, Western Blot and immunohistochemistry may be employed.

A particularly useful assay includes but is not limited to immobilized oligonucleotide-mediated detection-systems.

Generally, the effective amount of HBV genome required to be inserted into the baculovirus genome is functionally equivalent to but comprises more than 100% of an HBV genome. For example, constructs containing approximately 1.05, 1.1, 1.2, 1.28, 1.3, 1.4, 1.5 and 1.6-1.9, 2.0 and 3.0 times the HBV genome are particularly useful.

Any cells which are capable of infection by baculovirus may be used in the practice of the present invention. The hepatoblastoma cell line, HepG2, or its derivatives, is particularly useful and is capable of in vitro cell culture. Huh-7 cells may also be used. Alternatively, any permissive cell line such as but not limited to a hepatocyte cell line or a primary hepatocyte cell culture may be used.

For convenience, a genetic construct comprising an HBV genome and an infection effective amount of baculovirus genome is referred to herein as “HBV baculovirus”, “recombinant HBV baculovirus” and “HBV baculovirus vector”. Recombinant HBV baculovirus is an efficient vector for the delivery of HBV genetic information to human cells and can be used to initiate HBV gene expression and replication in the cells. HBV transcripts, intracellular and secreted HBV antigens are produced and replication occurs as evidenced by the presence of high levels of intracellular, replicative intermediates and protected HBV DNA in the medium. Covalently closed circular (CCC) DNA is present indicating that, in this system, HBV core particles are capable of delivering newly synthesized HBV genomes back into the nucleus of infected cells. Strong HBV gene expression can be detected as early as one day post-infection (p.i.) High levels of HBV replicative intermediates, extracellular DNA, and CCC DNA persist through at least 11 days p.i. Endogenous HBV enhancers and promoters may be used to obtain high levels of HBV expression and replication in the cells.

Reference to “HBV” or its “components” in relation to the detection assay includes reference to the presence of RNA, DNA, antigenic molecules or HBV-specific activities. Conveniently, the assay is conducted quantitively, partially quantitively or qualitively. Most preferably, total HBV RNA or DNA is detected which provides an amount of RNA or DNA in the presence of a particular agent. When the HBV variant is more resistant to a particular agent relative to a wild-type strain, then a graphical representation of total RNA or DNA versus concentration of agent is likely to result in a reduced gradient of inhibition and/or an increase in the concentration of agent required before inhibition of RNA or DNA generation.

Another aspect of the present invention contemplates a method for detecting an HBV agent which exhibits inhibitory activity to an HBV, said method comprising:
generating a genetic construct comprising a replication competent-effective amount of the genome from said HBV contained in a plasmid vector and then transfecting said cells with said construct;
contacting said cells, before, during and/or after transfection, with the agent to be tested;
culturing said cells for a time and under conditions sufficient for the HBV to replicate, express genetic sequences and/or assemble and/or release virus or virus-like particles if resistant to said agent; and
subjecting the cells, cell lysates or culture supernatant fluid to viral- or viral-component-detection means to determine whether or not the virus has replicated, expressed genetic material and/or assembled and/or been released in the presence of said agent.

In an alternative embodiment, the present invention provides a method for detecting an HBV agent which exhibits inhibitory activity to an HBV, said method comprising:
generating a genetic construct comprising a replication competent-effective amount of the genome from said HBV contained in or fused to an amount of a baculovirus genome effective to infect cells and then infecting said cells with said construct;
contacting said cells, before, during and/or after infection, with the agent to be tested;
culturing said cells for a time and under conditions sufficient for the HBV to replicate, express genetic sequences and/or assemble and/or release virus or virus-like particles if resistant to said agent; and
subjecting the cells, cell lysates or culture supernatant fluid to viral- or viral-component-detection means to determine whether or not the virus has replicated, expressed genetic material and/or assembled and/or been released in the presence of said agent.

In a further alternative embodiment of the present invention, there is provided a method for detecting an HBV agent which exhibits inhibitory activity to an HBV, said method comprising:
generating a continuous cell line comprising an infectious copy of the genome of said HBV in a replication competent effective amount such that said infectious HBV genome is stably integrated into said continuous cell line such as but not limited to 2.2.15 or AD;
contacting said cells with the agent to be tested;
culturing said cells for a time and under conditions sufficient for the HBV to replicate, express genetic sequences and/or assemble and/or release virus or virus-like particles if resistant to said agent; and
subjecting the cells, cell lysates or culture supernatant fluid to viral- or viral-component-detection means to determine whether or not the virus has replicated, expressed genetic material and/or assembled and/or been released in the presence of said agent.

When there is little or no evidence of HBV particles or viral components in the presence of an agent, then the agent is a candidate anti-HBV agent. The present invention also extends to screening for the effectiveness of a combination of two or more agents. The latter is useful for combination therapy.

The present invention provides, therefore, compositions comprising the agents identified by the aforementioned method. Furthermore, the present invention contemplates the use of the agents identified as above in the manufacture of a medicament for the treatment of HBV infection in a patient.

The ability to detect replication fitness variants of HBV and agents with a reduced likelihood of selecting for same enables improved therapeutic management of HBV infection.

Accordingly, another aspect of the present invention contemplates a method of treating a patient infected with HBV, said method comprising administering to said patient an effective amount of a nucleoside analogue sufficient initially to inhibit HBV replication, monitoring HBV levels to ascertain the presence of an increased viral load in the presence of said nucleoside analogue and then changing the therapeutic protocol to permit inhibition of HBV levels.

Although the present invention is particularly exemplified in relation to the development of HBV variants resistant to LAM, the present invention extends to any nucleoside analogue including chemical derivatives and homologues of LAM which can result in the development of a level of replication fitness in the presence of said nucleoside analogue similar to or greater than the levels in the absence of said nucleoside analogue.

In a preferred embodiment, the present invention contemplates a method of treating a subject infected with HBV, said method comprising administering to said subject an effective amount of LAM or its chemical derivatives or homologues or a functionally related nucleoside analogue for a time and under conditions sufficient for the development of HBV variants exhibiting levels of replication fitness in the presence of said nucleoside analogue similar to or greater than in the absence of said nucleoside analogue and resistance to HBIG or its equivalent and then altering the therapeutic protocol to enable the inhibition of replication of the HBV variants.

The term “inhibition of replication” is all encompassing and includes inhibiting, reducing or otherwise affecting one or more stages of infection including replication, assembly and/or release of HBV or its variants as well as any intermediary stages of viral replication, assembly or release.

The term “resistant” or its derivations such as “resistance” includes complete or partial resistance to an anti-HBV agent. Generally, resistance to LAM or its chemical derivatives or homologues means the development of HBV variants having a replication fitness similar to or greater than the replication fitness in the absence of the nucleoside analogue.

A “functionally related nucleoside analogue” to LAM is one which results in the development of HBV variants having a nucleoside analogue mediated replication fitness similar to or greater than the replication fitness in the absence of the nucleoside analogue.

Although the present invention is particularly directed to the development of nucleoside analogue mediated replication fitness variants following exposure to both a nucleoside analogue and HBIG, the present invention extends to the selection of such variants following exposure to either treatment separately. Furthermore, although HBIG generally refers to a commercially prepared immunoglobulin to HBV surface antigen, the term is to be considered to encompass other anti-HBV immunoglobulin preparations including an immune response by the patient.

Yet a further aspect of the present invention contemplates a method of treatment based on the likelihood or possibility of development of nucleoside analogue mediated replication fitness mutants. Accordingly to this aspect, there is provided a method of treating a subject infected in with HBV, said method comprising administering to said subject an anti-HBV agent or combination of agents which after prolonged exposure to said HBV does not select for nucleoside analogue mediated replication fitness HBV variants.

This aspect of the present invention extends, therefore, to both single agent therapy as well as combination therapy.

An assessment of a potential viral variant is important for selection of an appropriate therapeutic protocol. Such an assessment is suitably facilitated with the assistance of a computer programmed with software, which inter alia adds index values (Iv) for at least two features associated with the viral variants to provide a potency value (PA) corresponding to the resistance or sensitivity of a viral variant to a particular chemical compound or immunological agent. The Iᵥs can be selected from (a) the ability to exhibit resistance for reduced sensitivity to a particular compound or immunological agent; (b) an altered DNA polymerase from wild-type HBV; (c) an altered surface antigen from wild-type HBV; or (d) morbidity or recovery potential of a patient. Thus, in accordance with the present invention, Iᵥs for such features are stored in a machine-readable storage medium, which is capable of processing the data to provide a P_{A} for a particular viral variant or a biological specimen comprising same.

Thus, in another aspect, the invention contemplates a computer program product for assessing the likely usefulness of a viral variant or biological sample comprising same for determining an appropriate therapeutic protocol in a subject, said product comprising:
(1) code that receives as input Iᵥs for at least two features associated with said viral agents or biological sample comprising same, wherein said features are selected from:
   (a) the ability to exhibit resistance for reduced sensitivity to a particular compound or immunological agent;
   (b) an altered DNA polymerase from wild-type HBV;
   (c) an altered surface antigen from wild-type HBV; or
   (d) morbidity or recovery potential of a patient;
(2) code that adds said Ivs to provide a sum corresponding to a Pv for said viral variants or biological samples; and
(3) a computer readable medium that stores the codes.

In a related aspect, the invention extends to a computer for assessing the likely usefulness of a viral variant or biological sample comprising same in a subject, wherein said computer comprises:
(1) a machine-readable data storage medium comprising a data storage material encoded with machine-readable data, wherein said machine-readable data comprise Iᵥs for at least two features associated with said viral variant or biological sample; wherein said features are selected from:
   (a) the ability to exhibit resistance for reduced sensitivity to a particular compound or immunological agent;
   (b) an altered DNA polymerase from wild-type HBV;
   (c) an altered surface antigen from wild-type HBV; or
   (d) morbidity or recovery potential of a patient;
(2) a working memory for storing instructions for processing said machine-readable data;
(3) a central-processing unit coupled to said working memory and to said machine-readable data storage medium, for processing said machine readable data to provide a sum of said I_{V}s corresponding to a P_{V} for said compound(s); and
(4) an output hardware coupled to said central processing unit, for receiving said P_{V}.

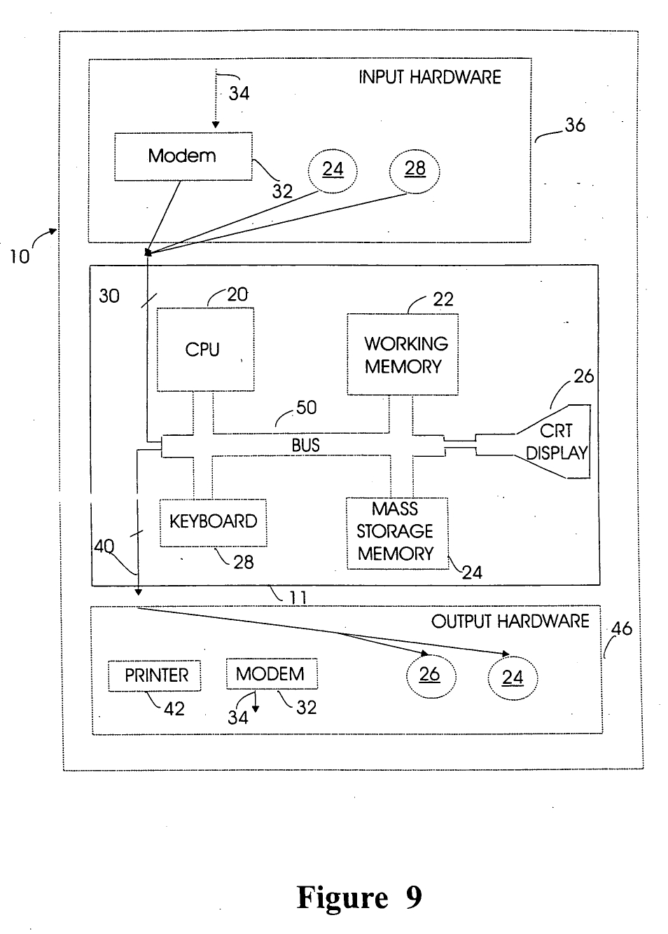

A version of these embodiments is presented in FIG. 9, which shows a system 10 including a computer 11 comprising a central processing unit (“CPU”) 20, a working memory 22 which may be, e.g., RAM (random-access memory) or “core” memory, mass storage memory 24 (such as one or more disk drives or CD-ROM drives), one or more cathode-ray tube (“CRT”) display terminals 26, one or more keyboards 28, one or more input lines 30, and one or more output lines 40, all of which are interconnected by a conventional bidirectional system bus 50.

Input hardware 36, coupled to computer 11 by input lines 30, may be implemented in a variety of ways. For example, machine-readable data of this invention may be inputted via the use of a modem or modems 32 connected by a telephone line or dedicated data line 34. Alternatively or additionally, the input hardware 36 may comprise CD. Alternatively, ROM drives or disk drives 24 in conjunction with display terminal 26, keyboard 28 may also be used as an input device.

Output hardware 46, coupled to computer 11 by output lines 40, may similarly be implemented by conventional devices. By way of example, output hardware 46 may include CRT display terminal 26 for displaying a synthetic polynucleotide sequence or a synthetic polypeptide sequence as described herein. Output hardware might also include a printer 42, so that hard copy output may be produced, or a disk drive 24, to store system output for later use.

In operation, CPU 20 coordinates the use of the various input and output devices 36,46 coordinates data accesses from mass storage 24 and accesses to and from working memory 22, and determines the sequence of data processing steps. A number of programs may be used to process the machine readable data of this invention. Exemplary programs may use, for example, the following steps:
(1) inputting input Iᵥs for at least two features associated with said compound(s), wherein said features are selected from:
   (a) the ability to exhibit resistance for reduced sensitivity to a particular compound or immunological agent;
   (b) an altered DNA polymerase from wild-type HBV;
   (c) an altered surface antigen from wild-type HBV; or
   (d) morbidity or recovery potential of a patient;
(2) adding the Iᵥs for said features to provide a Pᵥ for said compound(s); and
(3) outputting said Pᵥ.
   Thus, the input Iᵥs may be obtained either by reading them from storage or by receiving them directly in real-time as they are input.

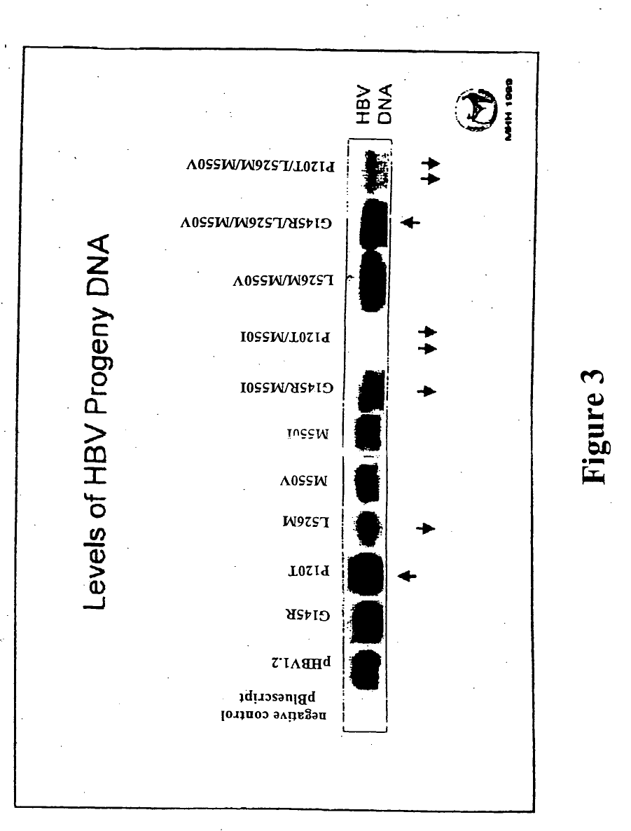

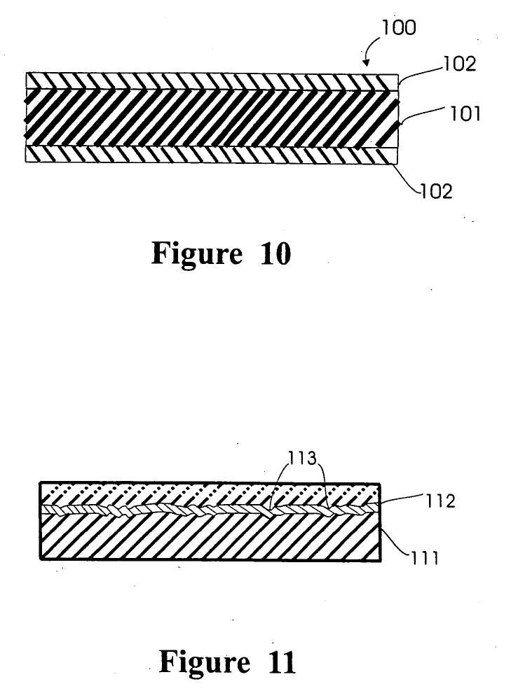

FIG. 10 shows a cross section of a magnetic data storage medium 100 which can be encoded with machine readable data, or set of instructions, for designing a synthetic molecule of the invention, which can be carried out by a system such as system 10 of FIG. 3. Medium 100 can be a conventional floppy diskette or hard disk, having a suitable substrate 101, which may be conventional, and a suitable coating 102, which may be conventional, on one or both sides, containing magnetic domains (not visible) whose polarity or orientation can be altered magnetically. Medium 100 may also have an opening (not shown) for receiving the spindle of a disk drive or other data storage device 24. The magnetic domains of coating 102 of medium 100 are polarised or oriented so as to encode in manner which may be conventional, machine readable data such as that described herein, for execution by a system such as system 10 of FIG. 9.

FIG. 11 shows a cross section of an optically readable data storage medium 110 which also can be encoded with such a machine-readable data, or set of instructions, for designing a synthetic molecule of the invention, which can be carried out by a system such as system 10 of FIG. 9. Medium 110 can be a conventional compact disk read only memory (CD-ROM) or a rewritable medium such as a magneto-optical disk, which is optically readable and magneto-optically writable. Medium 100 preferably has a suitable substrate 111, which may be conventional, and a suitable coating 112, which may be conventional, usually of one side of substrate 111.

In the case of CD-ROM, as is well known, coating 112 is reflective and is impressed with a plurality of pits 113 to encode the machine-readable data. The arrangement of pits is read by reflecting laser light off the surface of coating 112. A protective coating 114, which preferably is substantially transparent, is provided on top of coating 112.

In the case of a magneto-optical disk, as is well known, coating 112 has no pits 113, but has a plurality of magnetic domains whose polarity or orientation can be changed magnetically when heated above a certain temperature, as by a laser (not shown). The orientation of the domains can be read by measuring the polarization of laser light reflected from coating 112. The arrangement of the domains encodes the data as described above.

In the illustrated embodiment, the computer 11 is a desktop personal computer. However, the computer 11 may be implemented in virtually any type of electronic computing device such as a workstation, a laptop computer, a desktop computer, a mini-computer, a mainframe computer, or a supercomputer. The computer 11 may even be, in some alternative embodiments, a processor or controller embedded in a diagnostic tool, assuming the diagnostic tool has the requisite memory. The computer 11 may also be part of a larger computing system (not shown) such as a local area network (“LAN”), a wide area network (“WAN”), a system area network (“SAN”), an intranet, or even the Internet. In such an embodiment, the situs of the software components is not material to the practice of the invention. Thus, the data may be stored on one machine while it is processed on another machine, for example.

Note that some portions of the detailed descriptions herein are consequently presented in terms of a software implemented process involving symbolic representations of operations on data bits within a memory in a computing system or a computing device. These descriptions and representations are the means used by those in the art to most effectively convey the substance of their work to others skilled in the art. The process and operation require physical manipulations of physical quantities. Usually, though not necessarily, these quantities take the form of electrical, magnetic, or optical signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantifies. Unless specifically stated or otherwise as may be apparent, throughout the present disclosure, these descriptions refer to the action and processes of an electronic device, that manipulates and transforms data represented as physical (electronic, magnetic, or optical) quantities within some electronic device's storage into other data similarly represented as physical quantities within the storage, or in transmission or display devices. Exemplary of the terms denoting such a description are, without limitation, the terms “processing,” “computing,” “calculating,” “determining,” “displaying,” and the like.

Note also that, as was previously mentioned, the software implemented aspects of the invention are typically encoded on some form of program storage medium or implemented over some type of transmission medium. The program storage medium may be magnetic (e.g., a floppy disk or a hard drive) or optical (e.g., a compact disk read only memory, or “CD ROM”), and may be read only or random access. The invention is not limited by these aspects of any given implementation.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

### Overlapping Genome Of HBV

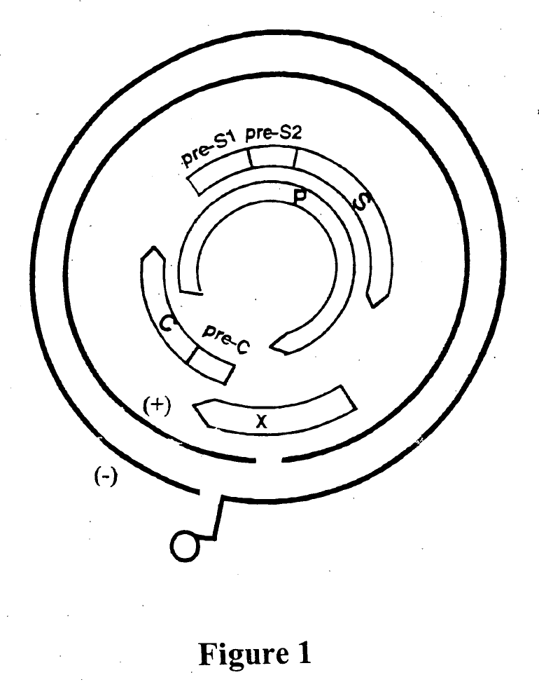

The overlapping genome of HBV is represented in FIG. 1. The gene encoding DNA polymerase (P), overlaps the viral envelope genes, Pre-S1 and Pre-S2, and partially overlaps the X and core (C) genes. The HBV envelope comprises small, middle and large HBV surface antigens. The large protein component is referred to as the HBV surface antigen (HBsAg) and is enclosed by the S gene sequence. The Pre-S1 and Pre-S2 gene sequences encode the other envelope components.

### EXAMPLE 2

### Assays

Northern Blots, antibodies and immunoprecipitation, viral DNA analogues, immunofluorescence and electron microscopy were as previously described (8,12).

### EXAMPLE 3

### Cell Culture

Sf21 insect cells were maintained in supplemented Grace's insect medium further supplemented with 10% vol./vol. heat-inactivated fetal bovine serum (Gibco BRL, Gaithersburg, Md.) in humidified incubator at 28° C. with CO₂. HepG2 cells were maintained in minimal essential medium supplemented with 10% v/v heat-inactivated fetal bovine serum (MEM-FBS). HepG2 cells were grown in humidified 37° C. incubators at 5% vol./vol. CO₂.

HuH-7 human heparoma cells were cultured in Dulbecco's modified Eagle medium supplemented with 10% vol./vol. fetal bovine serum under 5% wt./vol. CO₂ at 37° C. These cells are negative for HBV markers (14). DNA transfection into HuH-7 cells was performed as previously described (14). Cells were harvested from 2 to 6 days after transfection for analysis. Transfection efficiency was routinely checked by P-galactosidase assay. All transfection experiments were performed in duplicates or triplicates to verify the results.

### EXAMPLE 4

### Preparation of Baculovirus Transfer Vector

HBV plasmids are constructed in pBluescript KS+(Stratagene, San Diego, Calif.). This plasmid has been shown to be competent for HBV replication after transfection into HuH-7 cells (11).

Furthermore, a recombinant transfer vector is created by excising a fragment containing the required amount of variant HBV genome construct and cloning it into the multiple cloning region of a baculovirus vector such as pBlueBac4.5 (Invitrogen, Carlsbad, Calif.). Analysis of the recombinant transfer vector by restriction mapping demonstrates the presence of only one copy of the HBV genome portion in the construct. The nucleotide sequence of the plasmid and the point mutations generated by site directed mutagenesis are confirmed by sequencing using the ABI Prism Big Dye Terminator Cycle Sequencing Ready Reaction Kit according to the Manufacturer's specifications (Perkin Elmer, Cetus Norwalk, Conn.).

### EXAMPLE 5

### Generation of Recombinant Baculovirus

The HBV plasmid pHBV1.2 is used for mutational analysis. The pHBV1.2 contains a 1.28 mer HBV genome subtype adw2 in pBluescript II KS+.

### EXAMPLE 6

### HBV Variants

HBV mutants G145R, P120T, L526M, M550V and M550I were generated by site-directed mutagenesis (Stratagene). Transient transfection was achieved by the CaPO₄ precipitation with modifications as described by Chen and Okayama. Cell lines were HuH-7 and HepG2 hepatoma cells. Replicative intermediates for progeny DNA were isolated as described by Bock et al. (8). Standard procedures were used for Southern and Northern Blot experiments. Endogenous polymerase assays were performed as described earlier (8). Encapsidated RNA was isolated from immunoprecipitated HBV capsids and analyzed by Northern blotting. (+)-strand specific HBV probes were generated by a singled-stranded PCR method. Transfection efficiency was normalized by β-galactosidase assay. Experiments were generally conducted in triplicate.

### EXAMPLE 7

### Replication Fitness of HBV Polymerase Mutants

HBV variants were isolated from patients exhibiting increased viral loads a few months after treatment with lamivudine following OLT. The viral loads increased towards pre-treatment levels. Sequencing of the HBV genomes from the patients revealed mutations in the HBV polymerase gene, especially in the B- and C- domain of the polymerase. The inventors sought to determine the replication fitness of the HBV mutants selected during lamivudine therapy after liver transplantation.

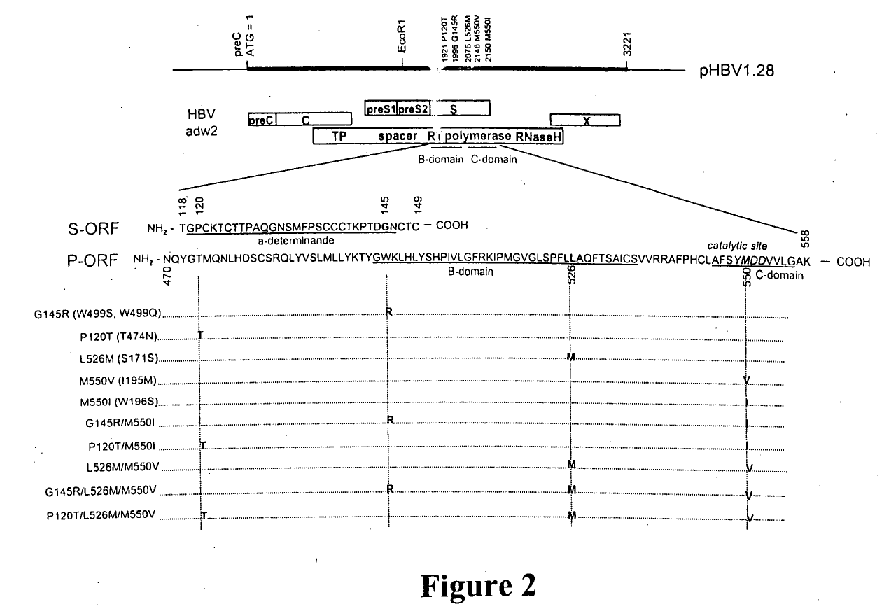

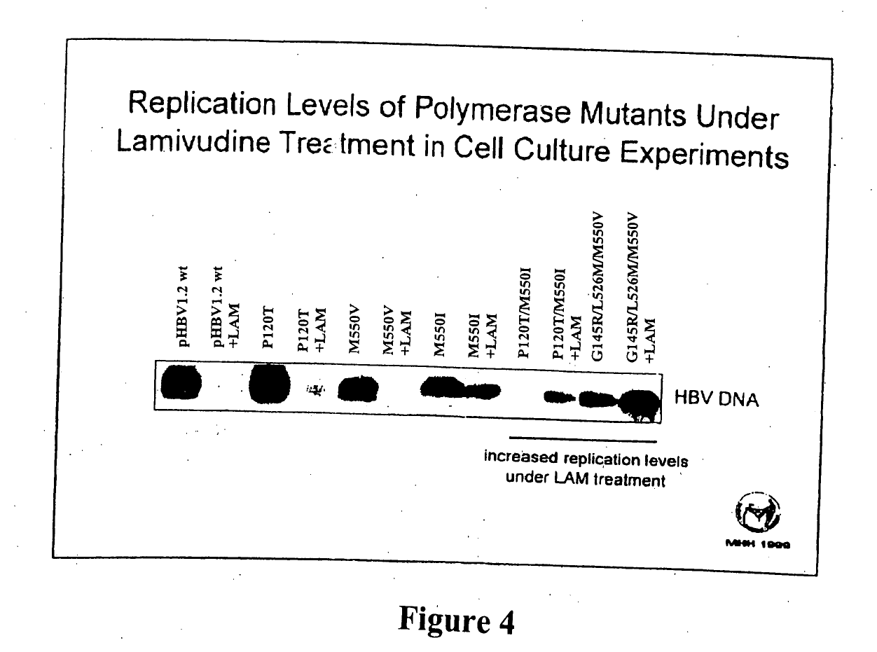

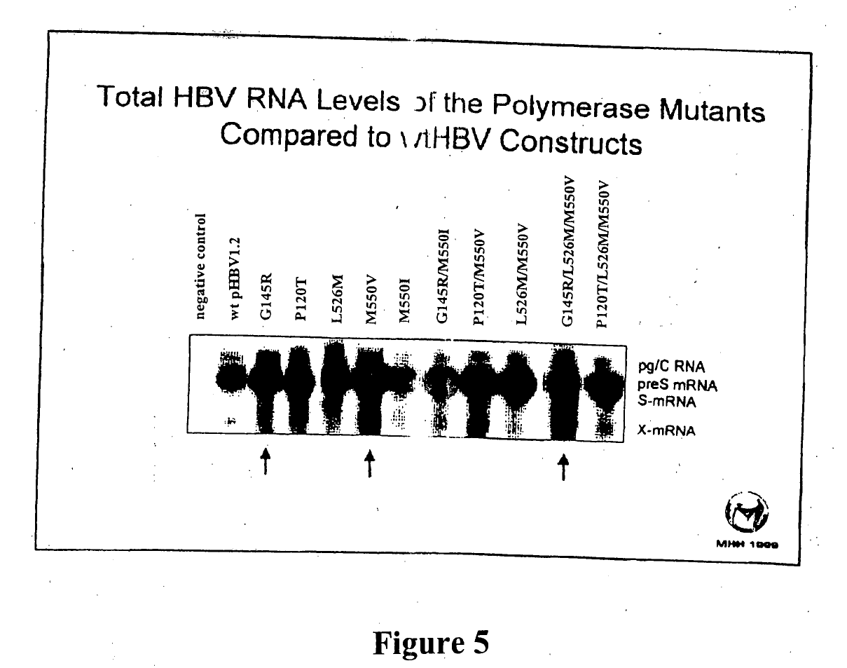

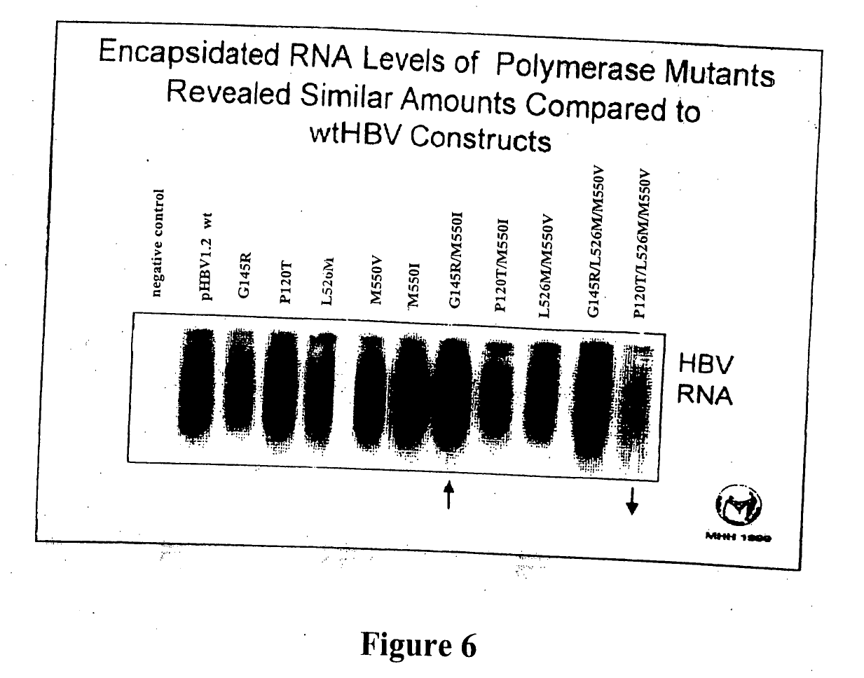

Point mutations found after OLT were: G145R, P120T, L528M, M550V and M550I. G145R is generally associated with HBIG immunoprophylaxis. The mutations associated with HBIG immunoprophylaxis are listed in FIG. 2. The above point mutations were introduced into a replication competent HBV vector pHBV1.2 (subtype adw2) alone or in combination using site directed mutagenesis and were transiently transfected into human hepatoma cells HuH-7 (FIG. 3). Cellular HBV RNA analysis revealed no difference between a wild-type construct and the different polymerase mutations (FIG. 4). Additionally, encapsidated HBV RNA showed comparable amounts of mutant and wild-type RNAs (FIG. 5 and FIG. 6). As expected, no change in the amount of protein levels were found if HBsAg was determined in the supernatant or cell lysate of infected cells. In another set of experiments, viral progeny DNA was examined for sensitivity to 3TC (5 μg/ml) and cells were harvested 5 days after transfection for analysis of encapsidated HBV DNA using immunoprecipitation methods and alkaline Southern Blot analysis. Mutants G145R, L526M and M550V showed replication levels comparable to wild-type constructs. P120T, M550I, P120T/M550I, G145R/M550I, G145R/L526M/M550V and P120T/L526M/M550V revealed reduced replication in comparison to wild-type HBV. Interestingly, if mutant P120T was transfected in combination with M550I or additionally with L526M as a triple mutant strain, HBV replication was strongly increased. Resistance or sensitivity to 3TC was shown for G145R, P120T, L526M, M550V, M550I, G145R/M550I and L526M/M550V. The mutants G145R/L526M/M550V, P120T/M550I and P120T/L526M/M550V were resistant to 3TC and showed higher replication levels in the presence of 3TC.

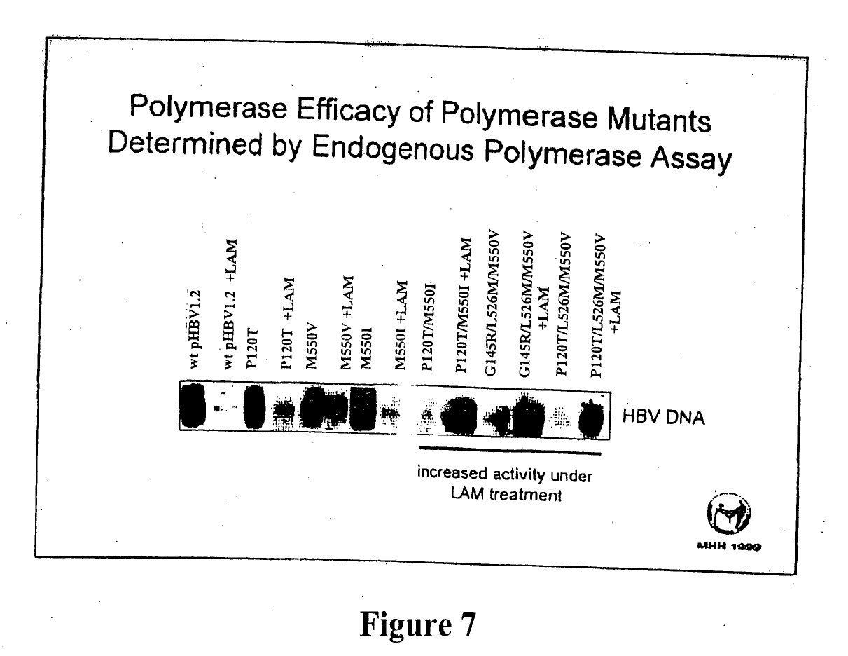

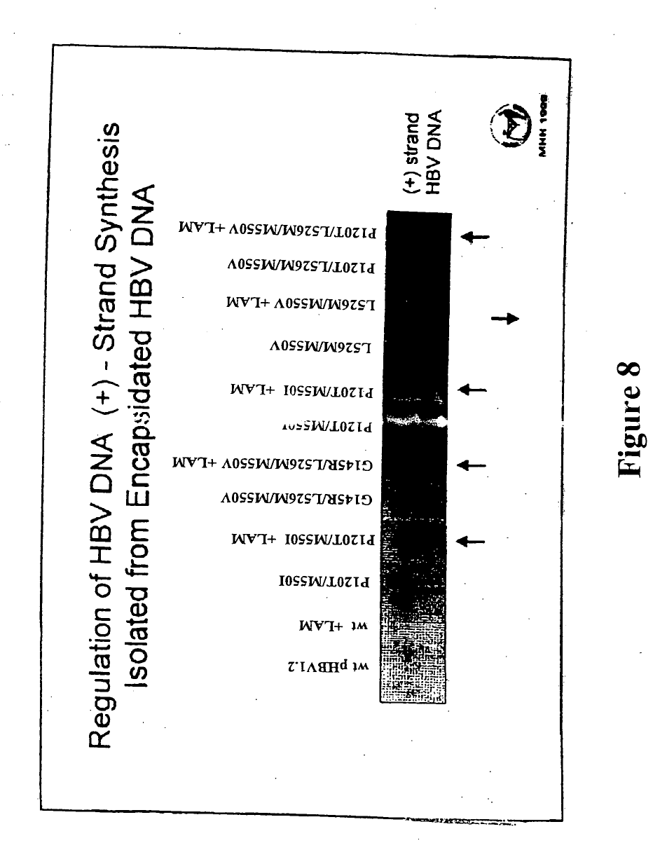

The results demonstrate that 3TC is a potential inhibitor of HBV replication by inhibiting the polymerase function of the virus (FIG. 7 and FIG. 8). However, a P120T mutant in combination with a mutation in the active region of the polymerase gene (B or C domain) is responsible for the severe clinical outcome of some patients with HBV-related liver disease because it results in a dramatic increase of HBV replication.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

1. Summers and Mason Cell 29:403-415, 1982
2. Vere Hodge Antiviral Chem Chemother 4:67-84, 1993
3. Boyd et al. Antiviral Chem Chemother. 32:358-363, 1987
4. Kruger et al. Hepatology 22:219A, 1994
5. Main et al. J Viral Hepatitis 3:211-215, 1996
6. Severini et al Antimicrobial Agents Chemother 39:1430-1435, 1995
7. Dienstag et al New England J Med 333:1657-1661, 1995
8. Bock et al. Gastroenterology 113:1976-1982, 1997
9. Nakbayashi et al. Cancer Res 42:3858-3863, 1982
10. Trautwein et al. Nature 364:544-547, 1993
11. Bruss and Ganem Proc. Natl. Acad. Sci USA 88:1059-1063, 1991
12. Tillmann et al Hepatology 30:244-256, 1999
13. Okamoto et al Paediatric Research 32:264-268, 1992
14. McMahon et al Hepatology 15:757-766, 1992
15. Norder et al. J. Gen. Virol. 74:341-1348, 1993
16. Poch et al. EMBO J. 8:3867-3874, 1989
17. Bartholomeusz et al. International Antiviral News 5:123-124, 1997

1-35. (canceled)

36. A method for detecting a candidate anti-HBV agent which exhibits inhibitory activity to an HBV, said method comprising the use of an HBV variant comprising a mutant DNA polymerase and optionally a mutant surface antigen (listed below in parenthesis) defined by T474N(P120T), M550V (1195M), M550I(W196S), L526M, W499S/W4990 (F145R), or combinations thereof with the proviso that the HBV variant does not contain an M550V or M550I mutation alone and wherein said HBV variant has a level of replication fitness in the presence of a nucleoside analogue similar to or greater than in the absence of said nucleoside analogue.

37. The method of claim 36 wherein the HBV variant is generated using a plasmid vector system or a baculovirus vector system.

38. The method of claim 36 comprising:
generating a genetic construct comprising a replication competent-effective amount of a genome from an HBV comprising a mutant DNA polymerase and optionally a mutant surface antigen (listed below in parenthesis) defined by T474N(P120T), M550V (1195M), M550I (W196S), L526M, W499S/W499Q (F145R), or combinations thereof which mutant is indicative of a variant which exhibits a replication fitness in the presence of a nucleoside analogue similar to or greater than in the absence of said nucleoside analogue with the proviso that the HBV does not contain an M550V or M5501 mutation alone, said genome contained in a plasmid vector and then transfecting cells with said construct;
contacting said cells, before, during and/or after transfection, with the agent to be tested;
culturing said cells for a time and under conditions sufficient for the HBV to replicate, express genetic sequences and/or assemble and/or release virus or virus-like particles if resistant to said agent; and
subjecting the cells, cell lysates or culture supernatant fluid to viral- or viral-component-detection means to determine whether or not the virus has replicated, expressed genetic material and/or assembled and/or been released in the presence of said agent.

39. The method of claim 36 comprising:
generating a genetic construct comprising a replication competent-effective amount of a genome from an HBV comprising a mutant DNA polymerase and optionally a mutant surface antigen (listed below in parenthesis) defined by T474N(P120T), M550V (1195M), M550I (W196S), L526M, W499S/W499Q (F145R), or combinations thereof which mutant is indicative of a variant which exhibits a replication fitness in the presence of a nucleoside analogue similar to or greater than in the absence of said nucleoside analogue with the proviso that the HBV does not contain an M550V or M5501 mutation alone, said genome contained in or fused to an amount of a baculovirus genome effective to infect cells and then infecting said cells with said construct;
contacting said cells, before, during and/or after infection, with the agent to be tested;
culturing said cells for a time and under conditions sufficient for the HBV to replicate, express genetic sequences and/or assemble and/or release virus or virus-like particles if resistant to said agent; and
subjecting the cells, cell lysates or culture supernatant fluid to viral- or viral-component-detection means to determine whether or not the virus has replicated, expressed genetic material and/or assembled and/or been released in the presence of said agent.

40. The method of claim 36 comprising:
generating a continuous cell line comprising an infectious copy of a genome of an HBV comprising a mutant DNA polymerase and optionally a mutant surface antigen (listed below in parenthesis) defined by T474N(P120T), M550V (1195M), M550I (W196S), L526M, W499S/W499Q (F145R), or combinations thereof which mutant is indicative of a variant which exhibits a replication fitness in the presence of a nucleoside analogue similar to or greater than in the absence of said nucleoside analogue with the proviso that the HBV does not contain an M550V or M550I mutation alone, said genome being present in a replication competent effective amount such that said infectious HBV genome is stably integrated in said continuous cell line such as but not limited to 2.2.15 or AD;
contacting said cells with the agent to be tested;
culturing said cells for a time and under conditions sufficient for the HBV to replicate, express genetic sequences and/or assemble and/or release virus or virus-like particles if resistant to said agent; and
subjecting the cells, cell lysates or culture supernatant fluid to viral- or viral-component-detection means to determine whether or not the virus has replicated, expressed genetic material and/or assembled and/or been released in the presence of said agent.

41. The method of claim 36 wherein the effectiveness of a combination of two or more candidate anti-HBV agents is determined.
